(19) United States
(12) Patent Application Publication
(10) Pub. No.: US 2007/0111205 A1
(43) Pub. Date: May 17, 2007

(54) Detection of somatic cells in milk

(76) Inventors: Micheal J. Delwiche, Winters, CA (US); Janice Wu, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/566,077

(22) PCT Filed: Jul. 16, 2004
(86) PCT No.: PCT/US04/22998

§ 371 Date: Sep. 26, 2006

Related U.S. Application Data
(60) Provisional application No. 60/490,126, filed on Jul. 25, 2003.
(60) Provisional application No. 60/556,243, filed on Mar. 24, 2004.

(51) Int. Cl. C12Q 1/68

(52) U.S. Cl. 435/6

(57) ABSTRACT

A simple, rapid and accurate method for detecting nucleic acids from milk are disclosed. The methods are useful in determining somatic cell count levels and diagnosing mastitis in cows.

### CROSS REFERENCE TO RELATED APPLICATIONS

This application claims priority to U.S. Provisional Application Ser. No. 60/556,243, filed Mar. 24, 2004 and U.S. Provisional Application Ser. No. 60/490,126, filed Jul. 25, 2004, both of which are herein incorporated by reference in their entirety for all purposes.

### BACKGROUND OF THE INVENTION

The single most costly disease of the dairy industry is mastitis, which is an inflammation of the mammary gland. Mastitis affects the dairy farmer financially through decreased milk yield, discarded milk, culling, drugs and veterinary expenses, and increased labor (DeGraves et al., Vet. Clinics N. Amer.: Food Animal Prac. 9:421, 1993; Blowey et al., Mastitis Control in Dairy Herds: An illustrated and practical guide 1995). The National Mastitis Council (1996) estimated the annual cost per cow to be $185, and the total annual cost of mastitis to be $1.8 billion. Blosser (J. Dairy Science 62:119, 1979) and Jasper et al. (21st Annual Mtg. Nat'l Mastitis Council 184, 1982) reported that the major cost of mastitis, which accounts for 65-70% of all costs, is reduction in milk yield. This expense is estimated to be $1 billion annually in the U.S. (Philpot et al., Counter Attack, 1991). The most costly form of the disease is subclinical mastitis because it is largely responsible for reduction in milk yield and quality (Fetrow, Compen. Contin. Educ. Prac. Vet. 11:223, 1980; Philpot et al., supra; Sandholm et al., The Bovine Udder & Mastitis, 1995).

Subclinical mastitis cannot be detected through visual inspection of the milk or udder, but can be detected through milk tests for an inflammatory response. One type of response is an increase in somatic cells. Somatic cells are 99% white blood cells and 1% epithelial cells. A study on cows enrolled in Dairy Herd Improvement (HI) in Wisconsin showed that each time a somatic cell count (SCC) doubles between 50 and 400 kcells/mL, milk yield decreases by 400 pounds (Philpot, Vet. Clin. N. Amer.: Large Anim. Prac. 6:233, 1984). Lightner et al. (J. Amer. Vet. Med. Assoc. 192:1410, 1988) reported that milk loss based on bulk tank SCC was responsible for 84% of total cost of mastitis on Ohio diary herds. California and federal regulations require individual producer milk to not exceed 750,000 cells/mL (Norman et al., J. Dairy Sci. 83:2782, 2000). The National Mastitis Council is more stringent and considers SCC below 100,000 cells/mL to be normal, and SCC above 200,000 to be abnormal and an indication of inflammation of the udder (Philpot et al., supra; Harmon, 40th Annual Mtg. Proc. Nat'l Mastitis Council 93, 2001). SCCs are now accepted as a standard measurement of raw milk quality by dairy industries worldwide (Dohoo et al., Canadian Vet. J. 23:119, 1982; Larry et al., NMC Guidelines 21, 2001). Consequently, SCCs are used to predict financial losses to dairy producers due to mastitis and the suitability of milk for human consumption and for downstream manufacturing of dairy products (Blowey et al., supra; Larry et al., supra).

The California Mastitis Test (CMT) and the Fossomatic (Foss Electric, Hillerod, Denmark) are currently used to provide information about SCCs. The CMT is based on the formation of a gel from the mixture of milk and a CMT reagent. The thickness of the gel is scored as 0, trace, 1, 2, or 3. The thicker the gel is, the higher the score. CMT scoring varies between testers and can only provide crude estimates of whether the count is high or low.

The Fossomatic is an automated flow cytometer that counts individual cells in a sample. The DNA of each cell is labeled with ethidium bromide and when excited, emits light at a certain wavelength. Each dyed cell produces an electric pulse, which can be measured and recorded automatically. The Fossomatic 5000 can run up to 500 samples an hour and has a repeatability of 4% when tested at 500,000 cells/niL.

Fossomatic analysis and often time the CMT are completed at remote sites where transportation of milk is required and results are not obtained until a couple days later. Time and effort is wasted to collect and ship samples and milk in transport risks degradation and contamination.

Thus, there remains a need in the art for simple, accurate, and efficient methods of detecting mastitis. The present invention fulfills these and other needs.

### BRIEF SUMMARY OF THE INVENTION

Given the above-mentioned issues of high cost, great effort and wasted time related to milk collection and transportation, it is the object of the present invention to provide a method of determining SCC levels in a fast, efficient and low cost manner. Advantages of the present invention include simple and quick detection of quarters and/or cows with high SCCs, thereby improving milk quality.

In one aspect, the present invention provides methods of detecting a nucleic acid in a milk sample. The method includes contacting the milk sample with a metal ion chelator. The milk sample is also contacted with a detergent. After contacting the milk sample with a metal ion chelator and a detergent, the nucleic acid is detected. Detecting the nucleic acid in the milk sample is thereby accomplished.

In another aspect, the present prevention provides a method to detect and/or quantify somatic cells in milk using a labeled nucleic acid marker and a sensor.

In another aspect, the present invention provides an analytical composition useful in detecting a nucleic acid in a milk sample and/or quantitating a nucleic acid thereby determining the somatic cell count within the milk sample. The analytical composition includes a milk sample, a metal ion chelator, and a detergent. The milk sample includes a nucleic acid.

In another aspect, the present invention provides a kit for practicing a method set forth herein. In an exemplary embodiment, the kit includes one or more components useful to practice the method of the invention and instructions for using that component to practice the method of the invention.

### BRIEF DESCRIPTION OF THE DRAWINGS

FIG. 1 illustrates a cross sectional view of an exemplary sensor.

FIG. 2 illustrates signal conditioning stages of a sensor.

FIG. 3 illustrates calibration of a sensor using ctDNA in buffer (V=201.8C+124.0, s_{V,C}=28.7 mV).

FIG. 4 illustrates calibration of a sensor using method one with raw milk, showing standard deviations (V=1.062C+60.5, s_{V,C}=132.4 mV).

FIG. 5 illustrates calibration of a sensor using method two with raw milk, showing standard deviations (V=0.325C+141.5, s_{V,C}=54.9 mV).

FIG. 6 illustrates calibration of a sensor using method three with raw milk, showing standard deviations (V=0.434C+802.9, s_{V,C}=20.2 mV).

FIG. 7 illustrates calibration of the sensor based on method one, with a 95% prediction interval based on 20 triplicate samples in the linear region (all data below 1000 kcell/ml) (v=1.73c+133, s_{vc}=146, r²=0.906).

FIG. 8 illustrates calibration of the sensor based on method three, with a 95% prediction interval based on 35 triplicate samples (v=0.291c+906, s_{vc}=118, r²=0.808).

FIG. 9 illustrates comparison of Foss and true SCC, with standard error and a 95% confidence interval based on 35 triplicate samples (f=1.01c+17, s_{fc}=233, r²=0.918).

FIG. 10 illustrates method three logistic regression for the probability of SCC above 200 kcell/ml.

FIG. 11 illustrate method three logistic regression for the probability of SCC above 750 kcell/ml.

### DETAILED DESCRIPTION OF THE INVENTION

Introduction

The present invention provides simple, rapid, and accurate methods of determining somatic cell levels in milk for use in the diagnosis of mastitis. The methods include a novel approach to detecting and/or quantitating nucleic acid in a milk sample. In addition, a sensor is provided that is useful in detecting and/or quantifying the nucleic acid.

Definition

Unless defined otherwise, all technical and scientific terms used herein generally have the same meaning as commonly understood by one of ordinary skill in the art to which this invention belongs. Generally, the nomenclature used herein and the laboratory procedures described below are those well known and commonly employed in the art. Generally, reactions and purification steps' are performed according to the manufacturer's specifications. Standard techniques, or modifications thereof, may be used for chemical analyses.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore or another moiety.

As used herein “fluorescent label”, refers to any atom or molecule which can be used to provide a detectable fluorescent signal, and which can be attached to a nucleic acid.

An organism is defined as having “mastitis” or “subclinical mastitis” when the somatic cell count within a milk sample is greater than or equal to 200 kcell/ml and bacteria are isolated in the absence of clinical changes.

A “crude milk sample” is a milk sample in which the milk fat is not removed from the raw sample (e.g., by centrifugation).

An “extraction mixture,” as used herein, refers to milk that has been treated according to the methods of the present invention for detecting nucleic acid in milk prior to detection of the DNA.

### DETAILED DESCRIPTION

The present invention provides methods of detecting a nucleic acid in a milk sample. In an exemplary embodiment, the nucleic acid is DNA. The DNA detected in the milk sample is indicative of the presence of somatic cells. Thus, quantitation of the DNA may be correlated to the somatic cell count (SCC) within the milk sample using methods known in the art and disclosed herein (see Examples below). The SCC may then be used to determine whether the organismic source of the milk is inflicted with mastitis (see Blowey et al., supra; Larry et al., supra). The present invention also provides a sensor that is calibratable in the physiological range using any known concentration of dsDNA (e.g. calf thymus DNA (ctDNA)). The sensor can be used to identify milk with SCCs in any range.

The methods of detecting nucleic acid in a milk sample include contacting the milk sample with a metal ion chelator. The milk sample is also contacted with a detergent. After contacting the milk sample with a metal ion chelator and a detergent, nucleic acid is in the milk sample is detected. Detecting the nucleic acid in the milk sample is thereby accomplished. In an exemplary embodiment, the milk sample is a bovine milk sample. In another exemplary embodiment, the nucleic acid is DNA.

The DNA may be detected using a detectable DNA probe. The DNA detection may be in the form of quantitation. Once the DNA is quantitated, the somatic cell count within the milk sample may then be determined.

The milk sample may be a purified milk sample, a partially purified milk sample, or a crude milk sample. Methods of purifying or partially purifying a milk sample are well known in the art and include, for example, column chromatography (e.g., size exclusion column chromatography, ion exchange column chromatography and the like), centrifugation, crystallization (including salting methods), organic solvent extraction, gel chromatography, and the like. A crude milk sample is a sample in which the milk fat is not removed from the raw sample (e.g., by centrifugation). In an exemplary embodiment, the milk sample is a crude milk sample. The milk sample may be from any appropriate source. In a related embodiment, the milk sample is a crude bovine milk sample.

In some embodiments, no proteases are added to the milk sample in the methods of the present invention to detect nucleic acids.

A variety of metal ion chelators are useful in the methods of the present invention. Exemplary metal ion chelators include chelators of divalent cations, such as calcium, magnesium, zinc, and manganese. Other useful metal ion chelators include, for example, EDTA (ethylenediamine-N,N,N′,N′,-tetraacetic acid), CyDTA (trans-1,2-diaminocyclohexane-N,N,N′,N′-tetraaceticacid), DHEG (N,N-Bis(2-hydroxyethyl)glycine), DTPA (1,3-diaminopropane-N,N,N′,N′-tetraacetic acid), EDDA (ethylenediamine-N,N′-diacetic acid), EDDP (ethylenediamine-N,N′-dipropionic acid dihydrochloride), EDDPO (ethylenediamine-N,N′-bis(methylenephosphonic acid)), EDTPO (ethylenediamine-N,N,N′,N′-tetrakis(methylenephosponic acid)), EGTA (O,O′-bis(2-aminoethyl)ethyleneglycol-N,N,N′,N′-tetraacetic acid), HBED (N,N-bis(2-hydroxybenzyl)ethylenediamine-N,N-diacetic acid), HDTA (1,6-hexamethylenediamine-N,N,N′,N′-tetraacetic acid), HIDA (N-(2-hydroxyethyl)iminodiacetic acid), IDA (iminodiacetic acid), NTA (nitrilotriacetic acid), NTP (nitrilotripropionic acid), NTPO (nitrilotris(methylenephosphoric acid)), O-Bistren (7,19,30-trioxa-1,4,10,13,16,22,27,33-octaazabicyclo [11,1,11]pentatriacontane), TTHA (triethylenetetramine-N,N,N′,N″,N′″,N′″-hexaacetic), o-phenanthroline, dipicolinic acid, deferoxamine, TTA (3-(2-thienoly)-1,1,1-trifluoroacetone), BFTA (3-benzoyl-1,1,1-trifluoroacetone), NPPTA (3-naphthoyl-1,1,1-trifluoroacetone), fod (2,2-dimethyl-4-perfluorobutyoyl-3-butanone), bpy (2,2′-dipyridyl), phen(phenanthroline), salicylic acid, phenanthroline carboxylic acid, bipyridyl carboxylic acid, aza crown ethers, trioctylphosphine oxide, aza cryptands, citric acid, and salts and derivatives thereof.

In an exemplary embodiment, the metal ion chelator is selected from the group of EDTA, CyDTA, DHEG, DTPA-OH, DTPA, EDDA, EDDP, EDDPO, EDTA-OH, EDTPO, EGTA, HBED, HDTA, HIDA, IDA, Methyl-EDTA, NTA, NTP, NTPO, O-Bistren, and TTHA, o-phenanthroline, dipicolinic acid, and deferoxamine. In a related embodiment, the metal ion chelator EDTA.

Metal ion chelators may be used at any appropriate concentration. In some embodiments, the concentration of the metal ion chelator is selected from 0.5 mM to 2.0 M. In a related embodiment, the concentration of the metal ion chelator is in the range of 1.0 mM to 1.0 M. In another related embodiment, the concentration of the metal ion chelator is between 0.1 M and 0.8 M, inclusive. In another related embodiment, the concentration of the metal ion chelator is between 0.4 M and 0.6 M, inclusive. In another related embodiment, the concentration of the metal ion chelator is 0.5 M.

Detergents of use in the present invention include non-ionic detergents and ionic detergentss. Non-ionic detergents do not ionize in aqueous solutions. Exemplary non-ionic detergents include sodium deoxycholate, octylglucoside, digitonin, octaethyleneglycol mono n-dodecyl ether (C12E8), lubrol, polyoxyethylated octyl phenol (Triton X-100), Ethylphenolpoly-(ethyleneglycolether) (Nonidet P-40), [Octylphenoxy]polyethoxyethanol (Nonidet P-40 substitute), Polyoxyethylene Sorbitan Monooleate (polyoxyethylenesorbitamnonooleat) (Tween 80), polyoxyethylene sorbitan monolaureate (Tween-20), BRIG 35, dodecyl maltopyranoside, heptyl thioglucopyranoside, ethylenoxide and propylenoxide block-copolymer detergents such as Pluronic and Tetronic detergents available from BASF (e.g. Pluronic F-127 and Tetronic T1307), Isotridecyl(PEG-ether)₈ (also referred to as Genapol X-080), N-alkanoyl-N-methylglucamide detergents (e.g. the MEGA series detergent including MEGA 9 AND MEGA 10), and the like.

Ionic detergents include anionic detergents, cationic detergents, and amphoteric detergents. Useful anionic detergents include, for example, sodium dodecyl sulfate, cholate and deoxycholate, and the like. Exemplary cationic detergents include cetyltrimethyl-ammonium bromide (CTAB) and the like. Amphoteric detergents useful in the present invention include, for example, LysoPC, CHAPS, Zwittergent 3-14, and the like.

In an exemplary embodiment, the detergent used in the methods of the present invention is a non-ionic detergent. In a related embodiment, the non-ionic detergent is selected from octylglucoside, digitonin, C12E8, lubrol, Triton X-100, Nonidet P-40, Tween 80, Tween-20, BRIG 35, dodecyl maltopyranoside, heptyl thioglucopyranoside, Pluronic F-127, Genapol X-080, and MEGA 10. In another exemplary embodiment, the detergent is Tween-20.

The pH of the milk in which the DNA is detected may be maintained between 7.0 and 12.0. In a related embodiment, the pH of the milk is between 8.0 and 1 1.0. Other useful pH ranges include 8.0-9.0, 9.0-10.0, and 11.0-12.0. In an exemplary embodiment, the pH is approximately 8.0, 8.5, 9.0, 9.5, 10.0, 10.5, 11.0, 11.5, or 12.0.

Buffering agents may be used to maintain the appropriate pH of the milk. Suitable buffering agents have a buffering capacity sufficient to maintain a desired pH while not causing deleterious effects to the DNA in the sample. Useful buffering agents include, for example, acetate, borate, citrate, HEPES (N-2-hydroxyethylpiperazine-N′-2-ethanesulfonic acid), BES (N,N-bis[2-hydroxyethyl]-2-amino-ethanesulfonic acid), TES (N-tris[Hydroxymethyl]methyl-2-aminoethanesulfonic acid), MOPS (morpholine propanesulphonic acid), PIPES (piperazine-N,N′-bis[2-etane-sulfonic acid]), and MES (2-morpholino ethanesulphonic acid).

In an exemplary embodiment, the methods of the present invention further include the step of vortexing the milk sample. The milk sample is typically vortexed from I to 30 seconds. In an exemplary embodiment, the milk sample is vortexed from 5 to 10 seconds. In an exemplary embodiment, the milk sample is vortexed for 1, 2, 3, 4, 5, 6, 7, 8, 9, or 10 seconds.

Detectable DNA probes useful in the present invention typically bind specifically to DNA, either covalently or non-covalently. The present invention is not limited by the mechanism in which the detectable DNA probe binds to DNA. Thus, detectable DNA probes of the present invention include those probes that intercalate DNA, covalently bond to DNA, ionically bond to DNA, and/or hydrogen bond to DNA.

In some embodiments, the detectable DNA probe is a fluorescent label. Fluorescent labels have the advantage of requiring few precautions in handling, and being amenable to high-throughput visualization techniques. Preferred labels are typically characterized by one or more of the following: high sensitivity, high stability, low background, low environmental sensitivity and high specificity in labeling. Many fluorescent labels are commercially available and those of skill in the art will recognize how to select an appropriate fluorophore and, if not readily available commercially, will be able to synthesize the necessary fluorophore de novo or synthetically modify commercially available fluorescent compounds to arrive at the desired fluorescent label. In the present invention, any fluorescent label that binds nucleic acid may be used. In an exemplary embodiment, the fluorescent label binds only dsDNA.

A multitude of fluorescent detectable DNA probes are useful in the current invention, including ethidium bromide, propidium iodide, SYBR Green I and II, PicoGreen, and Hoechst 33258 Dye. Other useful fluorescent detectable DNA probes include xanthenes, such as fluoresceins, benzofluoresceins, naphthofluoresceins, eosins, erytbrosins, rosamines, rhodamines (e.g., tetramethylrhodamine, sulforhodamines such as TEXAS RED dye), or rhodols. Additional useful fluorophores include benzimidazoles, phenoxazines (e.g., resorufin, nile blue), ethidiums, propidiums, anthracyclines, mithramycins, acridines, actinomycins, styryl dyes, carbocyanines, merocyanines, coumarins (e.g. 7-amino-4-methylcoumarins), pyrenes, chrysenes, stilbenes, carbazines, porphyrins, anthracenes, naphthalenes (e.g. dansyl, 5-dimethylamino-1-naphthalenesulfonyl), salicylic acids, anthranilic acids, benz-2-oxa-1,3-diazoles (also known as benzofurazans) (e.g. 4-amino-7-nitrobenz-2-oxa-1,3-diazole), fluorescamine, dipyrrometheneboron difluorides, and dibenzopyrrometheneboron difluorides, derivatives thereof, and those found in Haugland, Handbook of Fluorescent Probes and Research Chemicals, Molecular Probes, Eugene, (1992). In an exemplary embodiment, the detectable DNA probe is PicoGreen.

In an exemplary embodiment, the detection of the target analyte is accomplished by quantitation. Detection by quantitation is typically accomplished by quantitating the detectable DNA probe. Quantitation of the detectable DNA probe may be accomplished by any appropriate technique. Techniques useful in quantitating detectable DNA probes include, for example, those based on gel electrophoresis (e.g., agarose or polyacrylamide gels), liquid chromatography (e.g. HPLC), photospectrometry, and mass spectrometry. Quantitation methods useful in the present invention may be based on a variety of detectable DNA probe properties, including, for example, fluorescence (see Haugland, Handbook of Fluorescent Probes and Research Chemicals, Molecular Probes, Eugene, (1992)), radioactivity, fluorescence resonance energy transfer (FRET), electrochemilluminescence, chemilluminescence, fluorescence polarization or fluorescence anisotropy, absorbance, and the like.

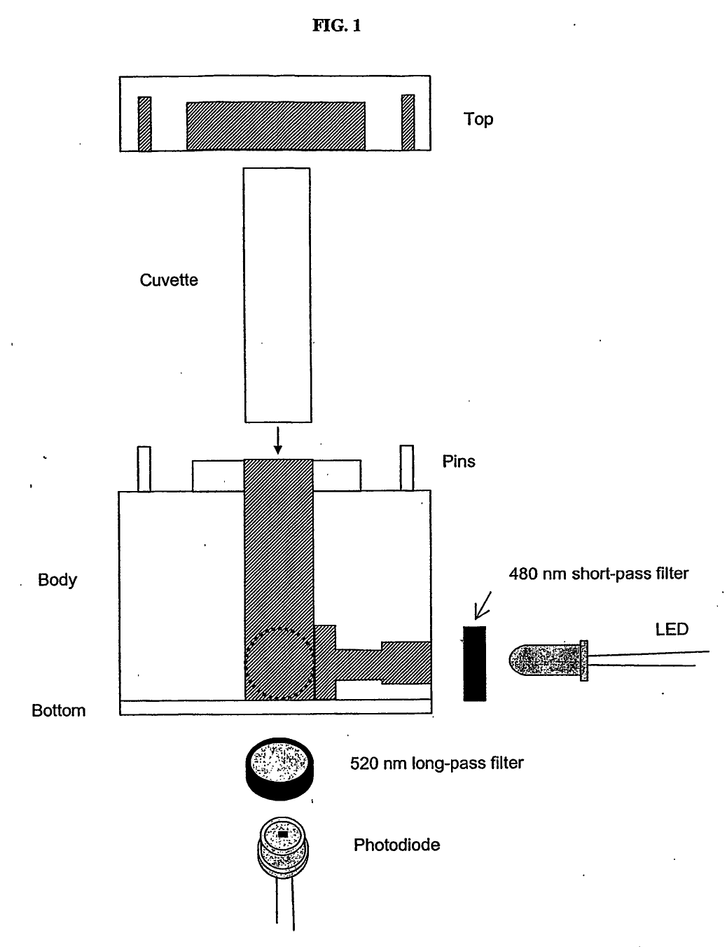

In some embodiments, a fluorescence detection system is used to detect and/or quantify nucleic acids. The fluorescence detection system may include a sensor and a circuit. The sensor may be housed in a casing. In some embodiments, the housing includes of a top, bottom and two sides, and contains an external source capable of exciting a fluorophore, a wavelength selection device for determining the excitation wavelength, a device for selecting the emission photons, and a detector that registers the emission photons as exemplified in FIG. 1. The above-mentioned devices will vary in excitation, filtration and detection according to the particular fluorescent marker used. The external source can be a light-emitting diode. The light-emitting diode may be capable of producing light at a wavelength of 470 nm. The wavelength selection device can be a short-pass edge filter. Preferably, it is a 480 nm short-pass edge filter. The device for selecting the emission photons can be a long-pass edge filter, for example, a 520 nm long-pass edge filter. The detection system may be a photodiode. The sensor components may be arranged such that the excitation photons pass through the sample and the emission photons are collected by the detection system.

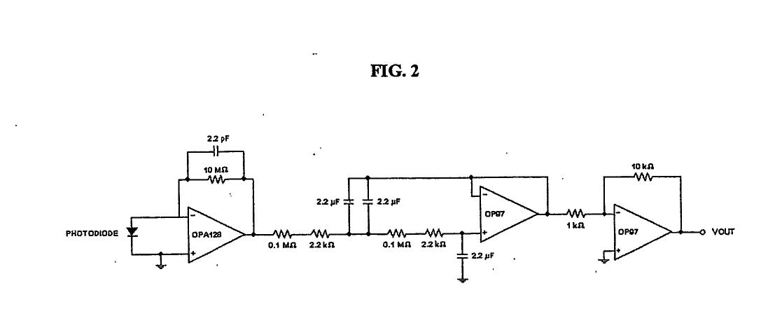

The circuit may be housed in a metal enclosure comprised of three stages, including a photovoltaic amplifier which can receive signals from the photodiode, a filter and a final gain stage using an inverted amplifier as exemplified in FIG. 2. The filter can be a low-pass filter. In some embodiments, the filter is a low-pass filter with a cutoff frequency of 0.5 Hz. Preferably, the final gain stage is such that the signal is amplified by a factor of 10.

The sensor may be calibrated using known quantities of nucleic acids, such as dsDNA. For calibrations, sensor output will generally start out in the same range for low DNA concentration and low SCCs. Calibration may have inflated outputs when opaque final DNA solutions are used in the sensor, attributable to light scattering and hence sensor output. Using methods known in the art, these values may be standardized based on opacity of the sample. Calibrations can be performed using a variety of methods and compared to optimize the invention for the particular conditions surrounding the use.

In some embodiments, the milk is diluted before calibration. The appropriate amount of dilution may be determined using the methods disclosed herein. In an exemplary embodiment, the extraction mixture is diluted between 2, 3, 4, 5, 6, 7, 8, 9, or 10 times its original volume.

In another aspect, the present invention provides an analytical composition useful in detecting a nucleic acid in a milk sample and/or quantitating a nucleic acid thereby determining the somatic cell count within the milk sample. The analytical composition includes a milk sample, a metal ion chelator, and a detergent. The milk sample includes a nucleic acid. Metal ion chelators, detergents, nucleic acids, and milk samples are described above and are equally applicable to the analytical compositions of the present invention. Typically, the analytical composition does not include a protease. The milk sample may be a crude milk sample.

In an exemplary embodiment, the nucleic acid is a DNA and the composition further includes a detectable DNA probe.

The metal ion chelator may be selected from EDTA, CyDTA, DHEG, DTPA-OH, DTPA, EDDA, EDDP, EDDPO, EDTA-OH, EDTPO, EGTA, HBED, HDTA, HIDA, IDA, Methyl-EDTA, NTA, NTP, NTPO, O-Bistren, and TTHA, o-phenanthroline, dipicolinic acid, and deferoxamine. In an exemplary embodiment, the metal ion chelator is EDTA.

The detergent is typically a non-ionic detergent, such as Octylglucoside, Digitonin, C12E8, Lubrol, Triton X-100, Nonidet P-40, Tween-80, Tween-20, BRIG 35, Dodecyl maltopyranoside, Heptyl thioglucopyranoside, Pluronic F-127, Genapol X-080, MEGA 10. In an exemplary embodiment, the detergent is Tween-20.

In some embodiments, the pH of the composition is between 8.0 and 11.0, inclusive. The pH may be maintained using a buffering agent as described above. In an exemplary embodiment, the pH of the composition is approximately 8.0, 8.5, 9.0, 9.5, 10.0, 10.5, 11.0, 11.5, or 12.0.

In another aspect, the present invention also provides a kit for practicing a method set forth herein. In an exemplary embodiment, the kit includes one or more components useful to practice the method of the invention and instructions for using that component to practice the method of the invention. Thus, the present invention provides a kit for detecting a nucleic acid in a milk sample and/or quantitating a nucleic acid thereby determining the somatic cell count within the milk sample. The kit includes a metal ion chelator and a detergent. Chemical components of the kit may be in dry form or in solution.

In an exemplary embodiment, the kit further includes a detectable DNA probe as described above. Where the DNA probe is a fluorescent DNA probe, the kit may also include a fluorescence detection system as described above and in the examples below. In some embodiments, a buffering agent may be included to maintain the pH of the sample solution at the desired pH.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, any feature of the methods of detecting or quantitating DNA in a milk sample may be incorporated into the kits or compositions of the present invention without departing from the scope of the invention.

In addition, the patents and scientific references cited herein are incorporated by reference in their entirety for all purposes.

### EXAMPLES

Materials and Methods

Chemical Reagents

PicoGreen (Molecular Probes, Eugene, Oreg., USA) was used as the fluorescent dsDNA marker because of its ultra sensitive binding to nonspecific dsDNA. ctDNA was purchased from Invitrogen Life Technologies (15633019, Carlsbad, Calif., USA). All other chemicals, including protease (P5147), were purchased from Sigma (St. Louis, Mo., USA). Milk samples were collected from the University of California, Davis dairy. Tests were conducted and samples were expressed-mailed to a DHI lab in Merced, Calif. on the same day of milk collection. The DHI lab used the Fossomatic 5000 (Foss, Denmark) to determine SCC. Candidate cows for milk sampling were chosen based on SCCs from monthly DHI reports.

### Example 1

### Assay Development

Three extraction procedures examples are provided. Method one included a fat removal step via centrifugation and the use of a commercial DNA extraction kit (DNeasy Tissue Kit, Cat. No. 69504, Qiagen, Valencia, Calif., USA). The DNA extraction method was performed as follows: Two milliliters of milk was centrifuged for 10 minutes at 5000 g. The fat and aqueous layer were removed. The pellet was resuspended in 200 μL of phosphate buffered saline. The steps for DNeasy Protocol for Cultured Animal Cells in the DNeasy Tissue Kit Handbook were then followed. In a cuvette, all of the final DNA solution (˜200 μL), 787 μL of 10 mM Tris, 1 mM EDTA, pH 7.5 (TE) and 13 μL of PicoGreen stock reagent were added. The cuvette was inverted to mix the solution and then inserted into the sensor and record the output voltage. Calibration with method one was the most sensitive to changes in SCC because the assay produced a purer form of DNA. The calibration equation based on method one could quantitate SCCs in the physiological range.

Methods two and three were assays designed for online application and did not contain DNA purification as in method one. However, calibrations with methods two and three identified milk with a low or medium SCC.

Method two included a fat removal step via centrifugation and cell lysis via mechanical shearing. In DNA extraction method two, two milliliters of milk were centrifuged at 5000 g for 10 minutes. The fat and aqueous layer were removed. The pellet was resuspended in 1 mL of 20 mM Tris, 300 mM NaCl, pH 9.0 and then vortexed for 10 seconds. 150 μL of 5 mg/mL protease was added and mixed by inverting. The suspension was incubated at 37° C. for 1 hour. The solution was syringe filtered through 5 μm and 1 μm filters. All of the filtered solution (˜950 μL), 37 μL of TE and 13 μL of PicoGreen was pipetted into a cuvette and mixed by inverting. The cuvette was inserted into the sensor and record the output voltage. Protease was added to eliminate DNases and milk proteins that contribute to sample opacity, and syringe filters (SLSVR25LS, FSLWO2500, Fisher Scientific, Pittsburgh, Pa., USA) were used to clarify the final DNA solutions. Method two conatined steps to protect DNA from degradation and reduce milk opacity, and therefore may result in more sensitive regression.

In method three, a detergent and mechanical shearing were used for cell lysis and a high pH was used to deactivate DNases and breakup casein micelles. In DNA extraction method three, the milk was diluted 1:10 with 10 mM Tris, 1 mM EDTA, pH 11.0. To 1 mL of milk was added 50 μL of 10% (w/v) Tween 20 and the mixture vortexed for 10 seconds. All of the final solution (˜1 mL) was transferred to a cuvette and 13 μL of PicoGreen was added. The final solution was then inverted to mix and inserted into the sensor and the output voltage recorded. In method three, no mechanical measures were taken to reduce sample opacity.

For all three methods, the final DNA solution was mixed with 13 μL of PicoGreen stock reagent and diluted with 10 mM Tris, 1 mM EDTA, pH 7.5 (TE) to bring the final volume to one mL. PicoGreen stock reagent was diluted approximately 1:100 in the final sample. Method three was the most preferred assay for online extraction of DNA because of its simplicity and rapid output.

### Example 2

### Sensor and Circuit Design

The sensor housing consisted of four pieces: the top, bottom, and two sides (FIG. 1). Side one contained a 470 nm light-emitting diode (LED) (BL-BBX3V4V-B02, American Bright, San Jose, Calif., USA) and a 480 nm short-pass edge filter (35-2039, Ealing Catalog, Inc., Rocklin, Calif., USA). Side two held a photodiode (BPW21R, Vishay, San Jose, Calif., USA) and a 520 nm long-pass edge filter (35-2153, Ealing Catalog, Inc., Rocklin, Calif., USA). The LED and photodiode were positioned perpendicular to each other. Sides one and two and the bottom piece were screwed together to form a block, which served as the main unit to hold a 1.5 mL sample cuvette (14-385-942, Fisherbrand, Fisher Scientific). The top piece completed the housing and was secured to the unit with pins.

The circuit consisted of three stages: a photovoltaic amplifier, a low-pass filter, and a final gain stage using an inverting amplifier (FIG. 2). An optical filter that passed wavelengths below 480 nm filtered light emitted from a LED. The filtered light excited PicoGreen and caused the dye bound to dsDNA to fluoresce at 520 nm. Fluorescence at wavelengths below 520 nm was filtered out with a 520 nm long-pass edge filter. The light was then detected by a photodiode, which produced a current proportional to the light intensity. The light current was sent to a photovoltaic amplifier with a gain of 106 V/A. The signal then passed through a 2nd order Butterworth low-pass filter with a cutoff frequency of 0.5 Hz. A final gain stage amplified the signal by a factor of 10. The circuit was housed in a metal enclosure for physical and electrical shielding purposes.

### Example 3

### Calibration

The sensor was calibrated with known concentrations of ctDNA in TE. Concentrations were prepared by diluting 500 ug/mL of ctDNA with TE to a final volume of 987 μL in a cuvette. The final solution was mixed with 13 μL of PicoGreen stock reagent by inversion. PicoGreen was given one minute to bind to ctDNA before the cuvette was inserted into the sensor for a reading. dsDNA was tested at concentrations between 0 to 6 μg/mL, which roughly corresponded to SCCs in the range of 0 to 1,000,000 cells/mL. Two replicates were completed at each concentration.

Preliminary calibrations were also completed with extracted DNA from raw milk with known SCCs using methods one, two, and three. Cows with low, medium and high levels of SCC were chosen based on monthly DHI reports. Based on the National Mastitis Council guidelines and federal and state regulations, SCCs less than or equal to 200,000 cells/mL were considered to be low, SCCs greater than 200,000, but less than or equal to 750,000 cells/mL were considered to be medium, and SCCs above 750,000 cells/mL were considered to be high. On the same day of milk collection, assays were conducted and milk samples were mailed to a DHI lab for estimates of SCCs. For calibrations with methods two and three, SCCs were determined based on triplicate milk samples. For calibration with method one, SCCs were based on a DHI report, since milk was collected on a DHI test day. Foss results from the lab were used as SCC standards. Three replicates of low, medium, and high milk were performed for all three methods.

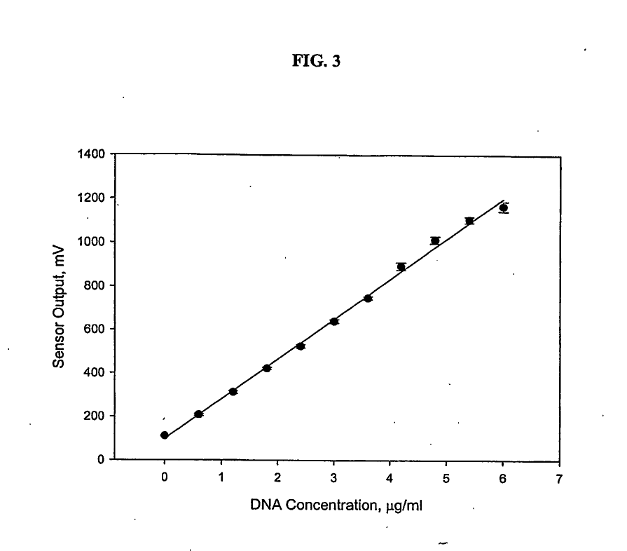

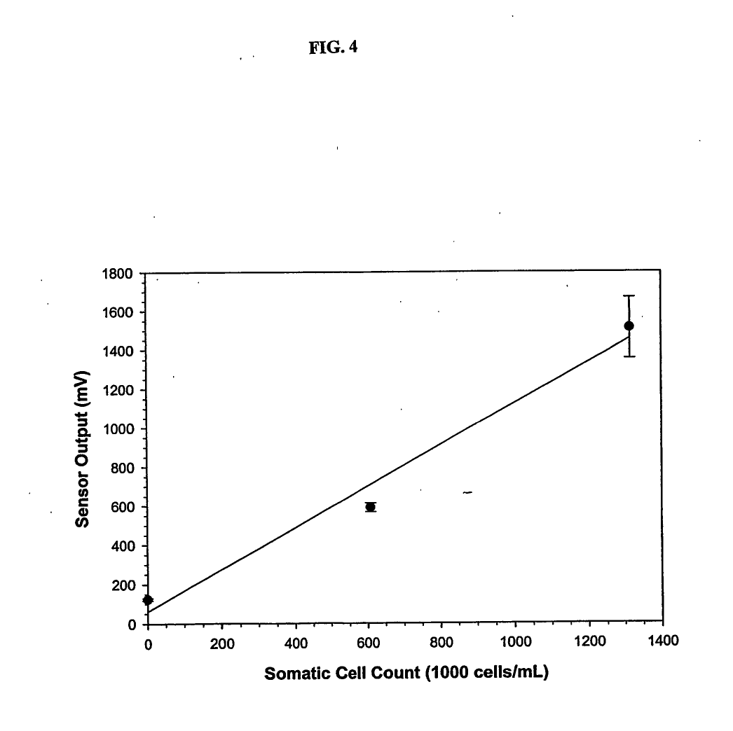

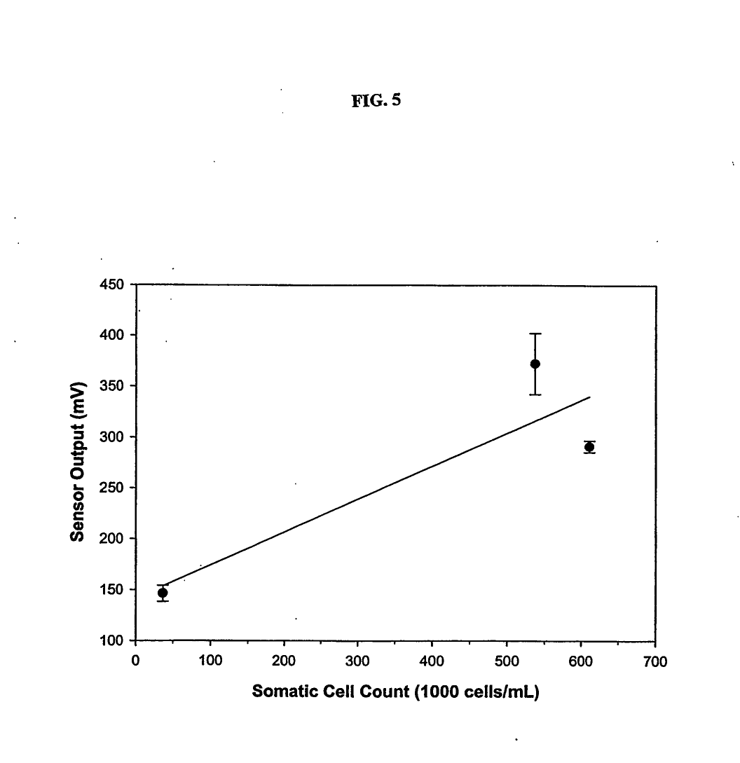

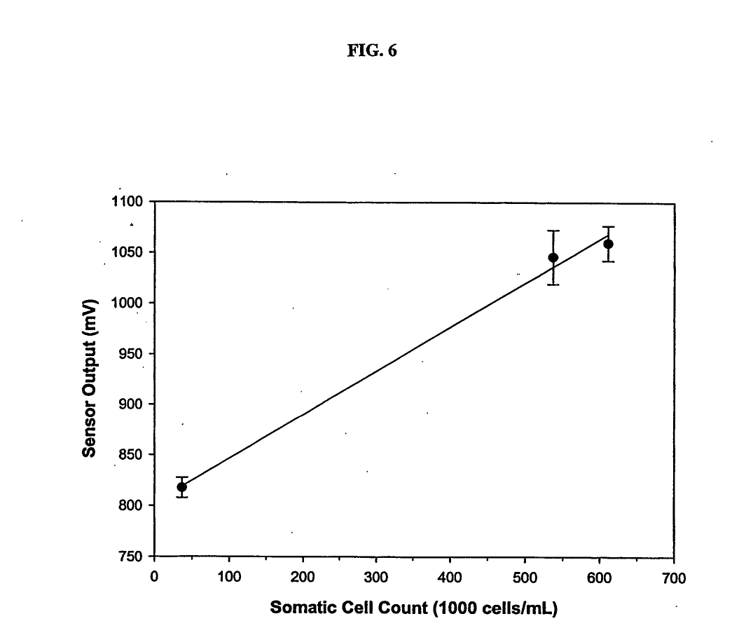

The sensor calibration with ctDNA in buffer showed a positive linear response between sensor output and DNA concentration, as exemplified in FIG. 3. Positive linear relationships were also obtained with calibration of the sensor using raw milk and methods one, two, and three, as exemplified in FIGS. 4, 5 and 6. For the calibration with raw milk and method one, the standard error of the sensor output estimate was 124,700 cells/mL and the calibration equation was:
C=0.942V−56.9 (1)
where C is the somatic cell count (1000 cells/mL) and V is the sensor output (mV).

For the calibration with ctDNA in buffer, a linear correlation was expected since dsDNA could be quantitated in the linear range as long as the reagent was in excess of the sample (Molecular Probes, 2003). This positive correlation verified that the sensor could produce larger output signals with samples of higher SCCs as shown in the calibrations with milk.

### Example 3.1

### Linear Detection Range

Further experiments were conducted to examine the saturated response and to develop a linear detection range calibration curve for methods one and three based on the saturated response.

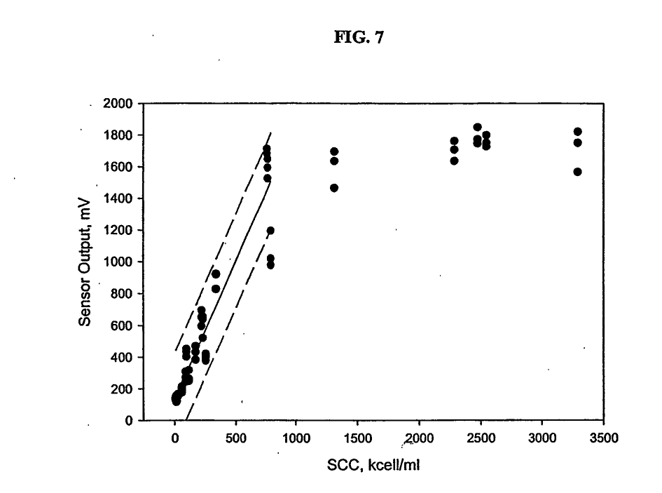

The calibration equations for DNA extracted by the methods were based on pooled results from two and three separate test days, respectively. The calibration of the sensor with DNA extracted from milk using method one produced a saturated response around 1 000 kcell/ml (FIG. 7), as expected. All points above 1000 kcell/ml were removed from the data set to develop a calibration equation in the linear detection range,
c=0.578v−77, (2)

where c is the concentration of cells in kcell/ml and v is the sensor output in mV. The standard error of prediction was 84 kcell/ml. The average coefficient of variation for the sensor output from triplicate readings of each sample was 6.1%.

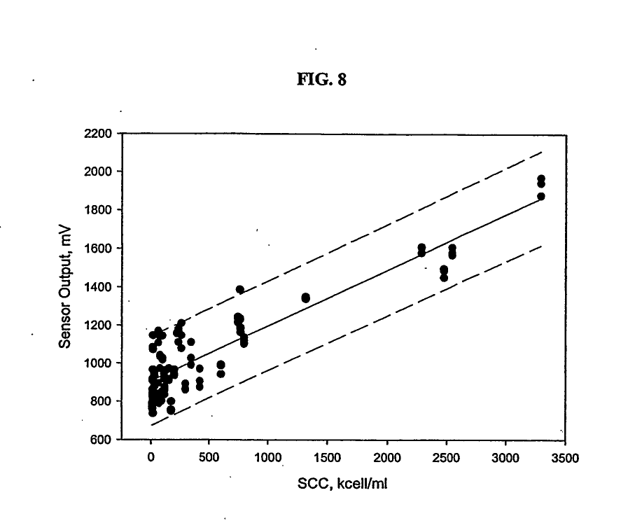

For method three, the output was still in the linear detection range at 3290 kcell/ml, verifying that saturation would not be an issue with this method (FIG. 8). The method three calibration equation based on SCC in the range of 10-3290 kcell/ml was
c=3.44v−3117, (3)
with a standard error of prediction of 406 kcell/ml. The average coefficient of variation for triplicate readings of each sample was 3.4%.

### Example 4

### Foss Count Comparison

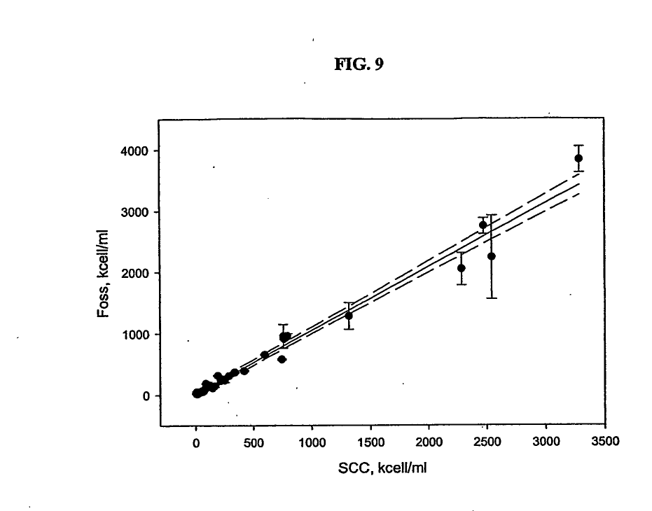

The Foss counts were compared to the microscopic counts to determine whether Foss counts were a reliable source for true SCC (FIG. 9). The mean Foss and microscopic counts were not found to be significantly different by an analysis of variance (ANOVA) at a significance level of 0.05. The root mean square error between the two methods was 232 kcell/ml. The average coefficient of variation for triplicate Foss readings of each milk sample was 14.5%. In general, the Foss counts tended to be higher than the direct microscopic somatic cell counts. When the data were separated into groups below 750 kcell/ml and above 750 kcell/ml, the root mean square error was larger for the high SCC group.

### Example 5

### Validation

The calibrated system was validated with two sets of milk samples, depending on the type of extraction used. Fifteen milk samples with SCC in the range of 5-79900 kcell/ml were used with the method three. Ten milk samples with SCC in the range of 20-745 kcell/ml were used with method one. Cell count classes were based on recommendations from the National Mastitis Council (NMC) and FDA guidelines. A cell count (in kcell/ml) less than 200 was classified as low, between 200 and 750 as medium, and above 750 as high. Error matrices were formulated to compare predictions for method three (Table 1) and method one (Table 2).

TABLE 1

| | Predicted | | | |
|---|---|---|---|---|
| True | Low | Med | High | Total |
| Low | 4 | 3 | 0 | 7 |
| Med | 0 | 0 | 4 | 4 |
| High | 0 | 2 | 2 | 4 |

TABLE 2

| | Predicted | | | |
|---|---|---|---|---|
| True | Low | Med | High | Total |
| Low | 5 | 1 | 0 | 6 |
| Med | 0 | 4 | 0 | 4 |
| High | 0 | 0 | 0 | 0 |

For method three, 57% of the low cases, 0% of the medium cases, and 50% of the high cases were classified correctly. For method one, 83% of the low cases and 100% of the medium cases were classified correctly. High SCC samples were not available to validate the high end of the method three calibrated system. Overall, 40% of the classifications were correct for method three, and 90% were correct for method three.

Where the system was used to predict only low and high counts with a threshold of 200 kcell/ml, 57% of the low cases and 100% of the high cases would be classified correctly. A system based on two classes would be useful for robotic milkers. When the cell count exceeded a threshold, a signal could be given to let the farmer know of a potential problem. The low occurrence of false positives is important.

Logistic regression was evaluated to determine whether SCC classifications based on method three would improve. Logistic regression fits probabilities for nominal dependent variables to a linear model of one or more continuous independent variables. For this study, a dichotomous response (0 or 1) was evaluated and p(v) was defined as the probability that the response was 1 for any v. Due to the constraint of p(v) between 0 and 1, a nonlinear transformation of the linear regression model was needed,

$$ln ( \frac{p ( v )}{1 - p ( v )} ) = a + bv . \qquad ( 4 )$$

The nonlinear transformation used was the natural log of the odds that the response was 1, which was assumed to change linearly with v. Logistic regressions were computed on sensor output to predict the probability of somatic cell counts exceeding 200 or 750 kcell/ml (JMP IN v4, SAS Institute, Cary, N.C., USA). The probability of achieving a SCC above 200 or 750 kcell/ml was determined from:

$$p ( v ) = \frac{1}{1 + {ⅇ}^{- ( a + bV )}} . \qquad ( 5 )$$

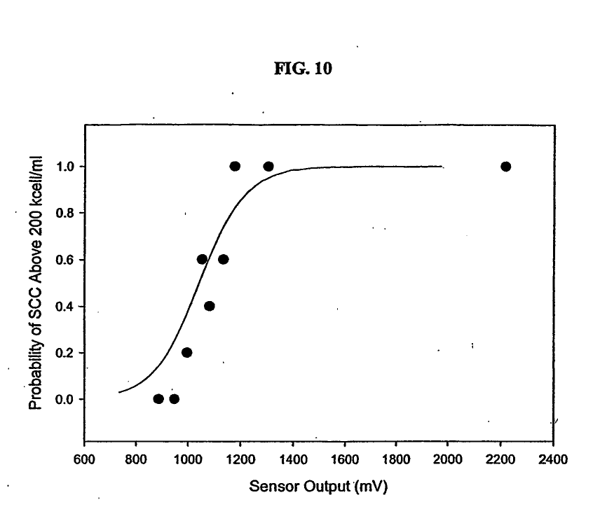

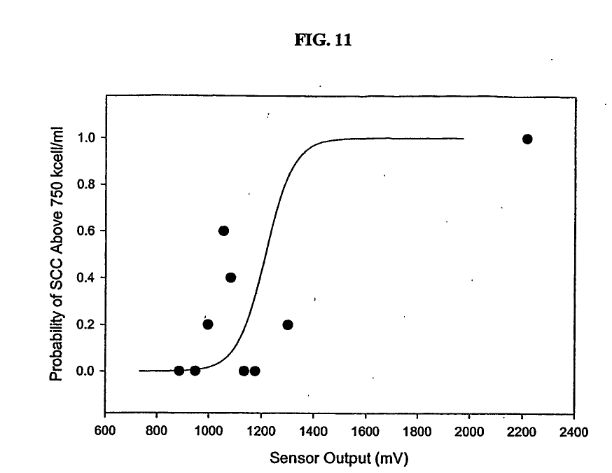

The logistic regression curves are shown in FIG. 10 and FIG. 11. The fraction of samples that had cells counts exceeding 200 or 750 kcell/ml are represented by the data points, and were found by grouping five successive values of sensor output, averaging the sensor output for the group, and finding the proportion of those with cell counts above 200 or 750 kcell/ml. The data used to develop the logistic regression models were based on samples collected on days one, two, and three. The p-values for both logistic regressions were less than 0.0001, indicating that the sensor output was significantly correlated to the probability of achieving SCC above 200 and 750 kcell/ml.

The logistic regression results were tested using samples collected on day four. Probabilities were calculated for each success level based on Eqn (5). When the probability was greater than 0.5, it was assumed that SCC was greater than the success cutoff. The results were organized into an error matrix to compare true and predicted counts (Table 3).

TABLE 3

| | Predicted | | | |
|---|---|---|---|---|
| True | Low | Med | High | Total |
| Low | 5 | 2 | 0 | 7 |
| Med | 0 | 3 | 1 | 4 |
| High | 1 | 1 | 2 | 4 |

The logistic regression classification was correct for 71% of the low cases, 75% of the medium cases, and 50% of the high cases. Overall, 67% of the classifications were correct. These classifications improved from the original classification, which was based on linear regression. For a low-high classification, 71% of the low cases (SCC less than 200) and 88% of the high cases (SCC above 200) were predicted correctly.

What is claimed is:

1. A method of detecting a DNA in a milk sample, said method comprising the steps of:
(a) contacting said milk sample with a metal ion chelator;
(b) contacting said milk sample with a detergent;
(c) after steps (a) and (b), detecting said DNA thereby detecting the DNA in said milk sample.

2. The method of claim 1, wherein no protease is added to said milk sample.

3. The method of claim 1, wherein said detecting said DNA is quantitating said DNA, thereby determining the somatic cell count within the milk sample.

4. The method of claim 3, wherein said milk sample is a crude bovine milk sample.

5. The method of claim 1, wherein said metal ion chelator is a member selected from the group of EDTA, CyDTA, DHEG, DTPA-OH, DTPA, EDDA, EDDP, EDDPO, EDTA-OH, EDTPO, EGTA, HBED, HDTA, HIDA, IDA, Methyl-EDTA, NTA, NTP, NTPO, O-Bistren, and TTHA, o-phenanthroline, dipicolinic acid, and deferoxamine.

6. The method of claim 1, wherein said metal ion chelator is EDTA.

7. The method of claim 1, wherein said detergent is a non-ionic detergent.

8. The method of claim 7, wherein said non-ionic detergent is a member selected from the group of Octylglucoside, Digitonin, C12E8, Lubrol, Triton X-100, Nonidet P-40, Tween-80, Tween-20, BRIG 35, Dodecyl maltopyranoside, Heptyl thioglucopyranoside, Pluronic F-127, Genapol X-080, MEGA 10.

9. The method of claim 1, wherein said detergent is Tween-20.

10. The method of claim 1, further comprising
(c) contacting said milk sample with a detectable DNA probe;
(d) after steps, (a), (b), and (c), detecting said detectable DNA probe thereby detecting said DNA in said milk sample.

11. The method of claim 1, wherein the pH of the milk sample is between 8.0 and 11.0, inclusive.

12. An analytical composition comprising a milk sample, a metal ion chelator, and a detergent, wherein said milk sample comprises a nucleic acid.

13. The composition of claim 12, wherein said milk sample is a crude milk sample.

14. The composition of claim 12, wherein said nucleic acid is a DNA.

15. The composition of claim 14, wherein said composition further comprises a detectable DNA probe.

16. The composition of claim 12, wherein said composition does not include a protease.

17. The composition of claim 12, wherein said metal ion chelator is a member selected from the group of EDTA, CyDTA, DHEG, DTPA-OH, DTPA, EDDA, EDDP, EDDPO, EDTA-OH, EDIPO, EGTA, HBED, HDTA, HIDA, IDA, Methyl-EDTA, NTA, NTP, NTPO, O-Bistren, and TTHA, o-phenanthroline, dipicolinic acid, and deferoxamine.

18. The composition of claim 12, wherein said metal ion chelator is EDTA.

19. The composition of claim 12, wherein said detergent is a non-ionic detergent.

20. The composition of claim 19, wherein said non-ionic detergent is a member selected from the group of Octylglucoside, Digitonin, C12E8, Lubrol, Triton X-100, Nonidet P-40, Tween-80, Tween-20, BRIG 35, Dodecyl maltopyranoside, Heptyl thioglucopyranoside, Pluronic F-127, Genapol X-080, MEGA 10.

21. The composition of claim 12, wherein said detergent Tween-20.

22. A kit for detecting a nucleic acid in a milk sample comprising a metal ion chelator, a detergent, and a detectable DNA probe.

23. The kit of claim 22 further comprising a fluorescence detection system.
